(19) United States
(12) Patent Application Publication
(10) Pub. No.: US 2003/0073889 A1
(43) Pub. Date: Apr. 17, 2003

(54) Monitoring led wavelength shift in photoplethysmography

(76) Inventors: Kevin A. Keilbach, Boulder, CO (US); Christina A. Knopp, Amesbury, MA (US)

Correspondence Address: MARSH, FISCHMANN & BREYFOGLE LLP, 3151 SOUTH VAUGHN WAY, SUITE 411, AURORA, CO, 80014, US

(21) Appl. No.: 09/975,807

(22) Filed: Oct. 11, 2001

(51) Int. Cl. A61B 5/00

(52) U.S. Cl. 600/322

(57) ABSTRACT

The present invention is directed to a method and system for the determination of a spectral characteristic of light signals emitted by light signal emitters of a photoplethysmographic probe to achieve improved accuracy in photoplethysmographic blood analyte level determinations. In one embodiment, a system for use in photoplethysmography includes a plurality of light signal emitters (20A-D) within a photoplethysmographic probe (12) and a voltage sensor (60) and data processor (70) within a photoplethysmographic monitor unit (14) to which the probe (12) is connectable. The voltage sensor (60) is operable to sense a voltage drop across each light signal emitter (20A-D) as each emits a corresponding light signal (22A-D) for transmission through a tissue under test. The data processor (70) is operable to establish at least one spectral characteristic, such as the center wavelength, of each emitted light signal (22A-D) based on the sensed voltage drops.

### FIELD OF THE INVENTION

The present invention generally relates to the field of photoplethysmography, and more particularly to the indirect monitoring of wavelength shifts in the light emitting diodes (LEDs) of a photoplethysmographic measurement probe to achieve improved accuracy in blood analyte level measurements.

### BACKGROUND OF THE INVENTION

Photoplethysmography involves the transmission of light signals through a tissue under test to non-invasively determine the level of one or more blood analytes. More specifically, photoplethysmographic devices are used to determine concentrations of blood analytes such as oxyhemoglobin (O2Hb), deoxyhemoglobin or reduced hemoglobin (RHb), carboxyhemoglobin (COHb) and methemoglobin (MetHb) in a patient's blood.

One type of photophlethysmographic device includes a probe having a plurality of light signal emitters, for example, four light emitting diodes (LEDs), and one detector. The probe is attachable to a patient's appendage (e.g. finger, ear lobe, nasal septum, foot) and is connectable via a cable with a monitor unit. The light signal emitters are operable to transmit light signals characterized by distinct center wavelengths λ_{A}≠λ_{B}≠λ_{C}≠λ_{D} through the patient's appendage to the detector. The monitor unit supplies drive signals via drive leads in the probe cable to the light signal emitters for turning the light signal emitters on and off as desired. The monitor unit also receives an output signal via an output lead in the cable from the detector indicative of the intensities of the transmitted light signals (light exiting the patient's appendage is referred to as transmitted). The monitor processes the output signal from the detector and, since different analytes have unique light absorbency characteristics, determines the concentrations of various blood analytes in the patient's blood based on the intensities of the transmitted light signals. See, e.g., U.S. Pat. No. 5,842,979.

The spectral characteristics of the light signal emitted by each light signal emitter may be dependent upon a number of factors, including the operating temperature of the emitter. For example, as the temperature of an LED changes, the width of the LED junction varies shifting the center wavelength of the light signal emitted by the LED. As may be appreciated, error may be introduced into the blood analyte level determinations if, when the determinations are made, it is assumed that the transmitted light signals have particular predetermined center wavelengths when, in fact, one or more of the transmitted light signals has a center wavelength differing from the predetermined assumed wavelength.

### SUMMARY OF THE INVENTION

Accordingly, the present invention provides for the determination of spectral characteristics, such as the center wavelengths, of the light signals emitted by the light signal emitters of a photoplethysmographic probe. The determined spectral characteristics may then be used to improve the accuracy of the determination of the concentrations of various blood analytes by a photoplethysmographic monitor unit. For example, information concerning the center wavelengths of the emitted light signals permits the monitor unit to adjust its calibration accordingly and thereby base its calculations on the actual center wavelengths of the emitted light signals rather than assumed center wavelengths.

According to one aspect of the present invention, a method for use in photoplethysmographic measurement of blood analyte levels includes the step of measuring a voltage drop across a light signal emitter as the light signal emitter emits a light signal for use in determining a blood analyte level. A spectral characteristic of the emitted light signal is then established based on the measured voltage drop across the light signal emitter. The established spectral characteristic of the emitted light signal is then used in determining a blood analyte level. In the method of the present invention, the light signal emitter may comprise an LED. The established spectral characteristic may comprise a center wavelength of the emitted light signal.

The method of the present invention may further include the step of obtaining data correlating the measured voltage drop across the light signal emitter with the spectral characteristic of the emitted light signal. The obtained data may be stored in a manner in which it can be used to establish the spectral characteristic of the emitted light signal in the establishing step of the method. In this regard, the data may comprise a plurality of pairs of data points. Each pair of data points includes a first value representing a voltage drop across the light signal emitter and a second value representing the spectral characteristic (e.g. center wavelength) of a corresponding light signal emitted by the light signal emitter. Such data may be used to look-up the spectral characteristic corresponding with the measured voltage drop across the light signal emitter in the establishing step of the method of the present invention. Since the measured voltage drop may not identically appear in the data, appropriate rounding or interpolation techniques may be employed in the establishing step to obtain the spectral characteristic of the emitted light signal from the stored data. The stored data may also comprise a slope and an intercept point of a plot of a measured voltage drop across the light signal emitter versus the spectral characteristic of a corresponding light signal emitted by the light signal emitter. In this regard, in the establishing step the spectral characteristic may be computed as the dependent variable of a linear equation described by the slope and intercept point and having the measured voltage drop as an independent variable.

The data may, for example, be obtained in the following manner. The light signal emitter may be operated a plurality of times to emit a plurality of light signals. When operating the light signal emitter, it is operated under varying operating conditions to thereby vary the spectral characteristics of the plurality of light signals output by the light signal emitter. For example, the temperature of an LED light signal emitter may be varied by heating and/or cooling the LED to vary its junction gap width and thereby vary the center wavelength of the light signals emitted by the LED. Each time the light signal emitter is operated the voltage drop across the light signal emitter is detected and the spectrum of the light signal emitted by the light signal emitter is analyzed to obtain the spectral characteristic of the emitted light signal.

According to another aspect of the present invention, a method of providing a spectral characteristic of a light signal emittable from a light signal emitter of a photoplethysmographic probe to a photoplethysmographic measurement unit includes the step of obtaining data correlating a voltage drop across the light signal emitter with the spectral characteristic of the light signal. The obtained data is stored in a manner in which it may be used to establish the spectral characteristic of the emitted light signal when the photoplethysmographic probe is used in determining a blood analyte level. As the light signal emitter is operated to emit the light signal, the voltage drop across the light signal emitter is measured. The stored data is then used to establish the spectral characteristic of the emitted light signal based on the measured voltage drop across the light signal emitter.

According to a further aspect of the present invention, a system for use in photoplethysmographic measurement of at least one blood analyte level in a tissue under test includes a plurality of light signal emitters (e.g., LEDs), a voltage sensor, and a data processor. In one embodiment, the light signal emitters may be disposed in a photoplethysmographic probe and the voltage sensor and data processor may be disposed in a photoplethysmographic monitor unit to which the photoplethysmographic probe is connectable. The light signal emitters are operable to emit a corresponding plurality of light signals for transmission through the tissue under test. The voltage sensor is operable to sense a voltage drop across each light signal emitter as each light signal emitter emits a corresponding light signal. The data processor is operable to establish at least one spectral characteristic of each emitted light signal based on the sensed voltage drop across the light signal emitter corresponding therewith. In this regard, the established spectral characteristic may be the center wavelength of each emitted light signal. The spectral characteristic established is usable in determining at least one blood analyte level.

The system of the present invention may also include a data storage device such as, for example, an erasable read only memory (EPROM) chip. The data storage device provides for storage of data correlating the sensed voltage drop across each light signal emitter with the spectral characteristic of each emitted light signal. In one embodiment, the data storage device may be disposed within the photoplethysmographic probe. The data is accessible to the data processor for use in establishing the spectral characteristic of each emitted light signal based on the sensed voltage drop across each light signal emitter corresponding therewith. In this regard, the data may comprise a plurality of pairs of data points. Each pair of data points corresponds with one of the light signal emitters and includes a first value representing a voltage drop across the corresponding light signal emitter and a second value representing the spectral characteristic of a light signal emitted by the corresponding light signal emitter. The data may also comprise a plurality of slope values and intercept points. Each slope value and intercept point is associated with a separate plot of the voltage drop across a corresponding one of the light signal emitters versus the spectral characteristic of the light signal emitted by the corresponding light emitter.

According to one more aspect of the present invention, a photoplethysmographic probe includes a plurality of light signal emitters and a data storage device (e.g., an EPROM chip). The photoplethysmographic probe is connectable with a photoplethysmographic monitor unit for use in photoplethysmographic measurement of at least one blood analyte level in a tissue under test. The light signal emitters are operable to emit a corresponding plurality of light signals for transmission through the tissue under test. The data storage device provides for storage of data correlating a sensed voltage drop across each light signal emitter when operated to emit a light signal with at least one spectral characteristic of each emitted light signal. The data is accessible to the monitor unit for use in establishing the spectral characteristic of each emitted light signal based on the sensed voltage drop across each light signal emitter.

These and other aspects and advantages of the present invention will be apparent upon review of the following Detailed Description when taken in conjunction with the accompanying figures.

### DESCRIPTION OF THE DRAWINGS

For a more complete understanding of the present invention and further advantages thereof, reference is now made to the following Detailed Description, taken in conjunction with the drawings, in which:

FIG. 1 is a diagrammatic illustration of one embodiment of a photoplethysmographic measurement apparatus in accordance with the present invention;

FIG. 2 depicts a table listing exemplary pairs of voltage drop and center wavelength data points for typical LEDs of the photoplethysmographic measurement apparatus of FIG. 1;

FIG. 3 shows plots of best-fit linear regression lines for the exemplary pairs of data points listed in the table of FIG. 2;

FIG. 4 shows one embodiment of an LED characterization system for use in obtaining the exemplary pairs of data points listed in the table of FIG. 2; and

FIG. 5 is a flow diagram illustrating the steps of one embodiment of a method for improving the accuracy of the determination of blood analyte levels by a photoplethysmographic monitor unit to which an interchangeable photoplethysmographic probe is connectable.

### DETAILED DESCRIPTION

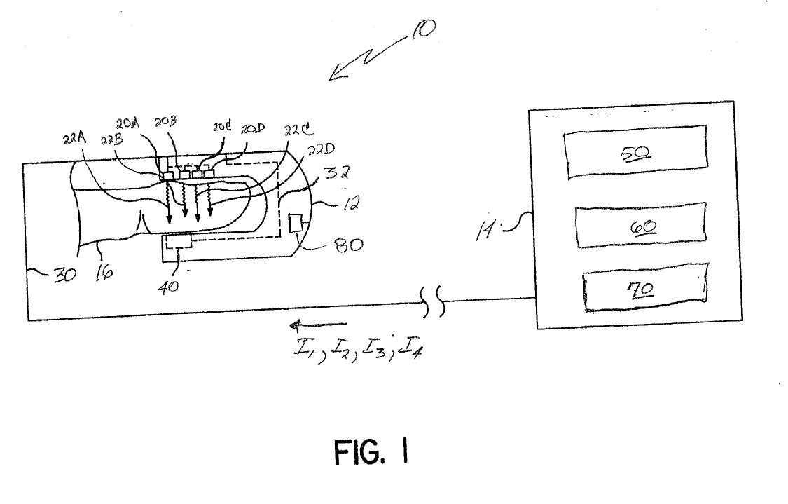

Referring now to FIG. 1, there is shown a diagrammatic illustration of one embodiment of a photoplethysmographic measurement apparatus 10 in accordance with the present invention. The photoplethysmographic measurement apparatus 10 is configured for use in determining one or more blood analyte levels in a tissue under test, such as O2Hb, RHb, COHb and MetHb levels. The apparatus 10 includes a probe 12 and a monitor unit 14. The probe 12 includes a plurality of LEDs 20A-D operable to emit a corresponding plurality of light signals 22A-D centered at different predetermined center wavelengths λ_{A}, λ_{B}, λ_{C}, λ_{D} through a tissue 16 under test (e.g., a person's finger) and on to a detector 40 (e.g., a photo-sensitive diode) within the probe 12. The center wavelengths λ_{A}, λ_{B}, λ_{C}, λ_{D} required depend upon the blood analytes to be determined. For example, in order to determine the levels of O2Hb, RHb, COHb and MetHb, λ_{A} may be about 640 nm, λ_{B} may be about 660 nm, λ_{C}may be about 800 nm, and λ_{D} may be about 940 nm. It should be appreciated that the present invention may be readily implemented with fewer or more LEDs depending upon the number of different blood analyte levels to be measured. The probe 14 facilitates alignment of the light signals 22A-D with the detector 40. In this regard, the probe 14 may be of a clip-type or a flexible strip configuration adapted for selective attachment to the tissue 16.

The LEDs 20A-D are activated by a corresponding plurality of drive currents I_{A}, I_{B}, I_{C}, I_{D} to emit the light signals 22A-D. The drive currents I_{A}, I_{B}, I_{C}, I_{D} are supplied to the LEDs 20A-D by a drive current generator 50 in the monitor unit 14 via a multi-conductor probe cable 30 connecting the probe 12 to the monitor unit 14. When each LED 20A-D is activated by its corresponding drive current I_{A}, I_{B}, I_{C}, I_{D}, there is an associated voltage drop V_{A}, V_{B}, V_{C}, V_{D} across the junction of the LED 20A-D. A voltage sensor 60 is provided in the monitor unit 14 for measuring the voltage drops V_{A}, V_{B}, V_{C}, V_{D} across each of the LEDs 20A-D. The voltage sensor 60 may, for example, measure the voltage drops V_{A}, V_{B}, V_{C}, V_{D} in the LEDs 20A-D via one or more sense wires within the multi-conductor probe cable 30.

The transmitted light signals 22A-D (i.e., the portions of light signals 22A-D exiting the tissue) are detected by the detector 40. The detector 40 detects the intensities of the transmitted light signals 22A-D and outputs a current signal, the level of which is indicative of the intensities of the transmitted light signals 22A-D. As may be appreciated, the current signal output by the detector 40 comprises a multiplexed signal in the sense that it is a composite signal including information about the intensity of each of the transmitted light signals 22A-D. Depending upon the nature of the drive currents, the current signal output from the detector 40 may, for example, be time-division multiplexed, wavelength-division multiplexed, code-division multiplexed, or a combination thereof. The current signal output by the detector 40 is directed to the monitor unit 14 via an output conductor 32 in the probe cable 30. The current signal from the detector 40, which may be amplified and filtered, is demultiplexed and processed by a processor 70 in the monitor unit 14 to determine the levels of one or more blood analytes. In this regard, the blood analyte levels may, for example, be determined as is disclosed in U.S. Pat. No. 5,842,979.

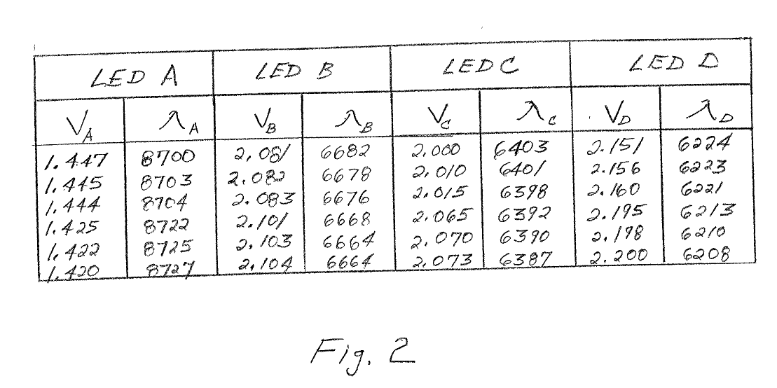

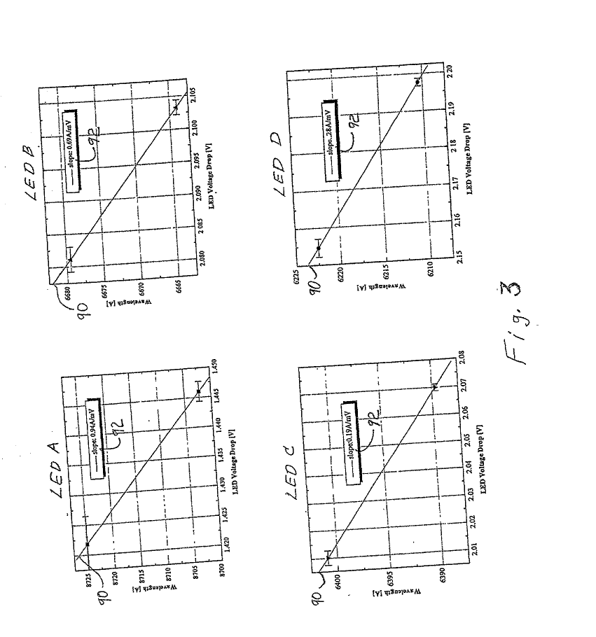

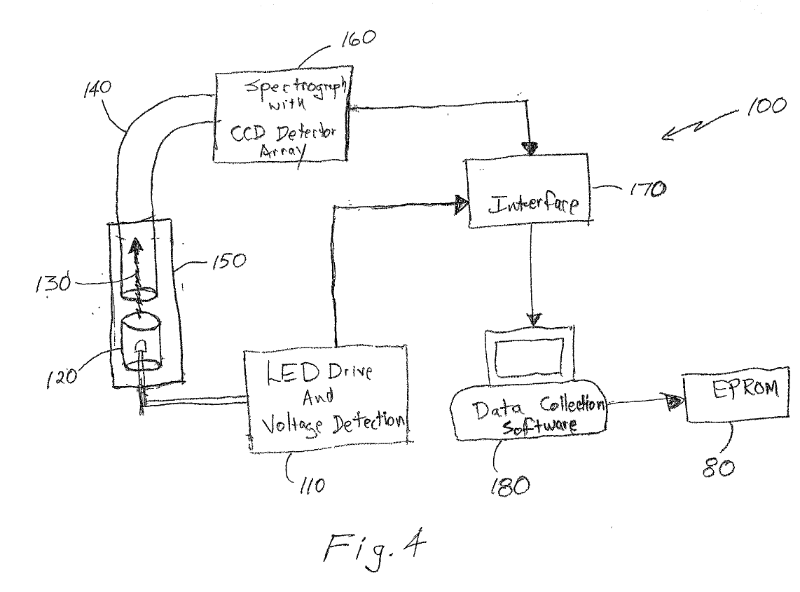

Also included in the probe 12 is an erasable programmable read only memory (EPROM) chip 80. Data correlating measured voltage drops across the LEDs 20A-D with spectral characteristics, such as the center wavelengths λ_{A}, λ_{B}, λ_{C}, λ_{D}, of the emitted light signals 22A-D is stored on the EPROM chip 80. More specifically, the data may comprise a number of pairs of data points corresponding with each of the LEDs 20A-D. Exemplary pairs of such data points for typical LEDs 20A-D are listed in the table shown in FIG. 2. As can be seen from the table of FIG. 2, each pair of data points corresponding with one of the LEDs 20A-D, has two values: (1) the voltage drop across the LED 20A-D; and (2) the center wavelength λ_{A}, λ_{B}, λ_{C}, λ_{D} of the light signal 22A-D emitted by such LED 20A-D. One manner of acquiring such data and a system for use in doing so is described below in connection with FIG. 4. As may be appreciated, the acquired data may be stored on the EPROM chip 80 in other manners. For example, statistical techniques (e.g., linear regression) may be applied to the data, and after doing so, the data may expressed and stored in a more compact manner. In this regard, the data may be stored on the EPROM chip 80 in the form of an intercept point 90 and slope 92 of the best-fit linear regression line for the acquired pairs of data values for each of the LEDs 20A-D as is shown in FIG. 3. It should be noted that as a result of the scaling of the plots shown in FIG. 3, the intercept points 90 are indicated on y-axes offset to the right from the origin rather than on y-axes passing through the origin. However, the intercept points 92 may be located on any appropriate y-axis including the y-axis passing through the origin.

Regardless of the form in which it is stored, the data on the EPROM chip 80 is made available to the processor 70 in the monitor unit 14 via a conductor in the probe cable 30. The processor may then use the measured voltage drops V_{A}, V_{B}, V_{C}, V_{D} across each of the LEDs 20A-D in conjunction with the data to determine the center wavelengths λ_{A}, λ_{B}, λ_{C}, λ_{D} of the emitted light signals 22A-D. For example, when all of the data values are stored on the EPROM chip 80 in the form illustrated in FIG. 2, the processor 70 may look-up the value for the center wavelength λ_{A}, λ_{B}, λ_{C}, λ_{D} of each light signal 22A-D by searching the table using the measured voltage drop V_{A}, V_{B}, V_{C}, V_{D} across its corresponding LED 20A-D provided to it by the voltage sensor 60. If necessary, appropriate rounding or interpolation techniques may be employed. If the data is stored in the form of intercept points 90 and slopes 92 of the voltage-versus-wavelength curves such as shown in FIG. 3, the processor 70 may use the measured voltage drops V_{A}, V_{B}, V_{C}, V_{D} to compute the center wavelength λ_{A}, λ_{B}, λ_{C}, λ_{D} of each light signal 22A-D in accordance with a linear equation described by the slopes 92 and intercept points 90. Once determined, the processor may use the determined center wavelengths λ_{A}, λ_{B}, λ_{C}, λ_{D} of the light signals 22A-D in its determination of various blood analyte levels instead of using assumed center wavelengths λ_{A}, λ_{B}, λ_{C}, λ_{D}, thereby achieving greater accuracy in the blood analyte determinations.

In the presently described embodiment, the LEDs 20A-D and EPROM chip 80 are included in the probe 12 and the voltage sensor 60 is included in the monitor unit 14. It should be appreciated that, in other embodiments, the LEDs 20A-D may be disposed within a connector body at the end of the probe cable 18 opposite the probe 14. In such embodiments, the light signals 30A-D emitted from the LEDs 20A-D may be directed from the LEDs 20A-D via one or more optical fibers in the probe cable 18 to the probe 12 for transmission through the tissue 16. Likewise, the EPROM chip 80 may also be disposed in the connector body rather than the probe 12. Such embodiments may provide for a more compact probe 12 by disposing the LEDs 20A-D and EPROM chip 80 in the connector body. Also, rather than being disposed in the monitor unit 14, the voltage sensor may be disposed within the probe 12 or a connector body at the end of the probe cable 18. The previously described embodiments are particularly suited to the situation wherein the probe 12 and monitor unit 14 are designed to be interchangeable (i.e., a number of probes 12 may be connected to a number of monitor units 14). In other embodiments where interchangeability is not desired, the LEDs 20A-D and EPROM chip 80 may be disposed within the monitor unit 14. Furthermore, the data need not be stored on an EPROM chip 80 within the probe 12 or monitor unit 14. For example, the data may be provided in a non-electronic manner (e.g., printed on a tag attached to the probe cable 18) and manually entered into the monitor unit 14 when the probe 12 is connected to the monitor unit 14.

Referring now to FIG. 4 there is shown one embodiment of an LED characterization system 100 for use in obtaining the data stored on the EPROM chip 80. The LED characterization system 100 includes an LED drive and voltage detection unit 110. The LED drive and voltage detection unit 110 provides a drive current to an LED 120 to activate the LED 120 for emission of a light signal 130. When the LED 120 is activated, the LED drive and voltage detection unit 110 measures the voltage drop across the LED 120. The emitted light signal 130 is directed via an optical fiber 140, which is disposed within an ambient light rejection fixture 150, to a spectrograph unit 160 having a charge-couple device (CCD) detector array. The CCD detector array of the spectrograph 160 detects spectral characteristics of the emitted light signal 130 directed thereto by the optical fiber 140. Each time that the LED 120 is activated by the LED drive and voltage detection unit 110, information regarding the spectrum of the emitted light signal 130 (e.g., its center wavelength) is provided by the spectrograph unit 160 via an interface 170 to a computer 180. Information regarding the measured voltage drop across the activated LED 120 corresponding to the emitted light signal 130 is also provided to the computer 180 from the LED drive and voltage detection unit 110 via the interface 170. The computer 180 is programmed with appropriate data collection software in order to collect the information regarding the spectrum of the light signal 130 and the corresponding voltage drop across the LED 120. The LED 120 may be activated a number of times under different operating conditions (e.g., by heating or cooling the LED 120 using an external heating/cooling source), in order to obtain a number of pairs of voltage drop and center wavelength values. The computer 180 may be programmed to process the collected data (e.g., to perform a linear regression of the data) to convert the data to a desirable format (e.g., a slope and intercept point format). Once the data is collected for the LED 120 and formatted as desired, it may be stored on the EPROM 90. It will be appreciated that the LED characterization system 110 may be configured to simultaneously characterize the spectrums of multiple LEDs 120 to be used in a photoplethysmographic probe.

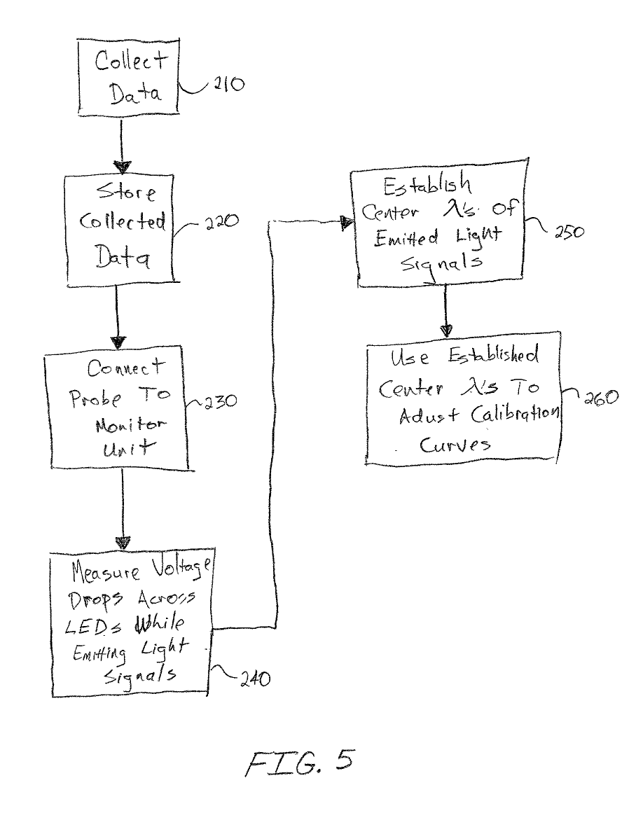

Referring now to FIG. 5, there is shown a flow diagram illustrating the steps of one embodiment of a method for improving the accuracy of the determination of blood analyte levels by a photoplethysmographic monitor unit to which an interchangeable photoplethysmographic probe is connectable. The method begins with step 210 wherein data correlating measured voltage drops across each LED in the photoplethysmographic probe with center wavelengths of light signals emitted by the LEDs is collected. One manner in which the data may be collected is through the use of an LED characterization system 110, such as shown in FIG. 4. In step 220, the collected data is stored on a data storage device (e.g., an EPROM chip) in the photoplethysmographic probe. In this regard, the data may be stored in a number of manners, such as in a table format as illustrated in FIG. 2 or in an intercept-slope format as illustrated in FIG. 3. Regardless of the format in which the data is stored, in step 230 the probe having the data stored therein is connected to the monitor unit thereby making the data available to the processor in the monitor unit.

In step 240 of the method, when the probe is activated to emit light signals from its LEDs through a tissue under test, the voltage drop across each emitting LED is measured by a voltage meter in the monitor unit. The measured voltage drops are then used to establish the center wavelength of each emitted light signal based on the measured voltage drop across its corresponding LED in step 250. In this regard, the processor may cross-reference the measured voltage drop across each emitting LED with the center wavelength using the data stored on the probe, or it may compute the center wavelength in accordance with an equation described by the data (e.g., a linear equation described by an intercept value and a slope). Once established, in step 260 the established center wavelengths of the emitted lights signals are used to adjust the calibration curves in the monitor unit prior to determining various blood analyte levels.

While various embodiments of the present invention have been described in detail, further modifications and adaptations of the invention may occur to those skilled in the art. However, it is to be expressly understood that such modifications and adaptations are within the spirit and scope of the present invention.

What is claimed is:

1. A method for use in photoplethysmographic measurement of blood analyte levels comprising the steps of:
measuring a voltage drop across a light signal emitter as the light signal emitter emits a light signal for use in determining a blood analyte level;
establishing a spectral characteristic of the emitted light signal based on the measured voltage drop across the light signal emitter; and
using the established spectral characteristic of the emitted light signal in determining a blood analyte level.

2. The method of claim 1 wherein the established spectral characteristic comprises a center wavelength of the emitted light signal.

3. The method of claim 1 wherein the light signal emitter comprises an LED.

4. The method of claim 1 further comprising the steps of:
obtaining data correlating the measured voltage drop across the light signal emitter with the spectral characteristic of the light signal emitted by the light signal emitter; and
storing the data wherein the data is usable to establish the spectral characteristic of the emitted light signal in said step of establishing a spectral characteristic of the emitted light signal.

5. The method of claim 4 wherein the data comprises a plurality of pairs of data points, each pair of data points including a first value representing a voltage drop across the light signal emitter and a second value representing the spectral characteristic of a corresponding light signal emitted by the light signal emitter.

6. The method of claim 4 wherein the data comprises a slope and an intercept point of a plot of a measured voltage drop across the light signal emitter versus the spectral characteristic of a corresponding light signal emitted by the light signal emitter.

7. The method of claim 4 wherein said step of obtaining comprises the steps of:
operating the light signal emitter a plurality of times to emit a plurality of light signals, wherein the light signal emitter is operated under varying operating conditions to vary the spectral characteristic of the plurality of light signals output by the light signal emitter;
detecting the voltage drop across the light signal emitter each time the light signal emitter is operated; and
analyzing the spectrum of each light signal emitted by the light signal emitter each time the light signal emitter is operated to obtain the spectral characteristic of each light signal emitted by the light signal emitter.

8. The method of claim 7 wherein the varying operating conditions include various temperatures.

9. The method of claim 4 wherein the light signal emitter is included in a photoplethysmographic probe including a data storage device and, in said step of storing, the data is stored on the data storage device of the probe.

10. The method of claim 9 wherein the data storage device comprises an EPROM.

11. A method of providing a spectral characteristic of a light signal emittable from a light signal emitter of a photoplethysmographic probe to a photoplethysmographic measurement unit wherein the spectral characteristic is usable in the determination of a blood analyte level by the photoplethysmographic measurement unit, said method comprising the steps of:
obtaining data correlating a voltage drop across the light signal emitter with the spectral characteristic of the light signal;
storing the data wherein the data is usable to establish the spectral characteristic of the emitted light signal when the photoplethysmographic probe is used in determining a blood analyte level;
measuring the voltage drop across the light signal emitter as the light signal emitter emits the light signal; and
using the stored data to establish the spectral characteristic of the emitted light signal based on the measured voltage drop across the light signal emitter.

12. The method of claim 11 wherein the spectral characteristic comprises a center wavelength of the emitted light signal.

13. The method of claim 11 wherein the light signal emitter comprises an LED.

14. The method of claim 11 wherein the data comprises a plurality of pairs of data points, each pair of data points including a first value representing a voltage drop across the light signal emitter and a second value representing the spectral characteristic of a corresponding light signal emitted by the light signal emitter.

15. The method of claim 11 wherein the data comprises a slope and an intercept point of a plot of a measured voltage drop across the light signal emitter versus the spectral characteristic of a corresponding light signal emitted by the light signal emitter.

16. The method of claim 11 wherein said step of obtaining comprises the steps of:
operating the light signal emitter a plurality of times to emit a plurality of light signals, wherein the light signal emitter is operated under varying operating conditions to vary the spectral characteristics of the plurality of light signals output by the light signal emitter;
detecting the voltage drop across the light signal emitter each time the light signal emitter is operated; and
analyzing the spectrum of the light signal emitted by the light signal emitter each time the light signal emitter is operated to obtain the spectral characteristic of the light signal emitted by the light signal emitter.

17. The method of claim 16 wherein the varying operating conditions include various temperatures.

18. The method of claim 11 wherein, in said step of storing, the data is stored on the data storage device of the probe.

19. The method of claim 18 wherein the data storage device comprises an EPROM.

20. A system for use in photoplethysmographic measurement of at least one blood analyte level in a tissue under test comprising:
a plurality of light signal emitters operable to emit a corresponding plurality of light signals for transmission through the tissue under test;
a voltage sensor operable to sense a voltage drop across each said light signal emitter as each said light signal emitter emits a corresponding light signal; and
a data processor operable to establish at least one spectral characteristic of each said emitted light signal based on the sensed voltage drop across said light signal emitter corresponding therewith, wherein said at least one spectral characteristic is usable in determining at least one blood analyte level.

21. The system of claim 20 wherein said at least one spectral characteristic comprises a center wavelength of each said emitted light signal.

22. The system of claim 20 wherein each said light signal emitter comprises an LED.

23. The system of claim 20 wherein said light signal emitters are disposed within a photoplethysmographic probe and said voltage sensor is disposed in a photoplethysmographic monitor unit to which said photoplethysmographic probe is connectable.

24. The system of claim 20 further comprising:
a data storage device for storing data correlating the sensed voltage drop across each said light signal emitter with said at least one spectral characteristic of each said emitted light signal, wherein said data is accessible to said data processor for use in establishing said at least one spectral characteristic of each said emitted light signal based on the sensed voltage drop across said light signal emitter corresponding therewith.

25. The system of claim 24 wherein said data comprises:
a plurality of pairs of data points, each said pair of data points corresponding with one of said light signal emitters and including a first value representing a voltage drop across said corresponding light signal emitter and a second value representing said at least one spectral characteristic of a corresponding light signal emitted by said corresponding light signal emitter.

26. The system of claim 24 wherein said data comprises:
a plurality of slope values and intercept points, each said slope value and intercept point being associated with a plot of the voltage drop across a corresponding one of said light signal emitters versus said at least one spectral characteristic of the light signal emitted by said corresponding light emitter.

27. The system of claim 24 wherein said data storage device is disposed within a photoplethysmographic probe and said data processor is disposed within a photoplethysmographic monitor unit to which said photoplethysmographic probe is connectable.

28. The system of claim 24 wherein said data storage device comprises an EPROM.

29. A photoplethysmographic probe connectable with a photoplethysmographic monitor unit for use in photoplethysmographic measurement of at least one blood analyte level in a tissue under test comprising:
a plurality of light signal emitters operable to emit a corresponding plurality of light signals for transmission through the tissue under test; and
a data storage device for storing data correlating a sensed voltage drop across each said light signal emitter when operated to emit a light signal with at least one spectral characteristic of each said emitted light signal, wherein said data is accessible to said monitor unit for use in establishing said at least one spectral characteristic of each said emitted light signal based on the sensed voltage drop across each said light signal emitter.

30. The photoplethysmographic probe of claim 29 wherein said at least one spectral characteristic comprises a center wavelength of each said emitted light signal.

31. The photoplethysmographic probe of claim 29 wherein each said light signal emitter comprises an LED.

32. The photoplethysmographic probe of claim 29 wherein said data comprises:
a plurality of pairs of data points, each said pair of data points corresponding with one of said light signal emitters and including a first value representing a voltage drop across said corresponding light signal emitter and a second value representing said at least one spectral characteristic of a corresponding light signal emitted by said corresponding light signal emitter.

33. The photoplethysmographic probe of claim 29 wherein said data comprises:
a plurality of pairs of slope values and intercept points, each said slope value and intercept point of a pair being associated with a plot of the voltage drop across a corresponding one of said light signal emitters versus said at least one spectral characteristic of the light signal emitted by said corresponding light emitter.

34. The photoplethysmographic probe of claim 29 wherein said data storage device comprises and EPROM.
